(19) United States
(12) Patent Application Publication
(10) Pub. No.: US 2024/0415379 A1
(43) Pub. Date: Dec. 19, 2024

(54) COMPUTER PROGRAM, INFORMATION PROCESSING METHOD, AND ENDOSCOPE

(71) Applicant: HOYA CORPORATION, Tokyo (JP)

(72) Inventor: Kyosuke MATSUMURA, Tokyo (JP)

(73) Assignee: HOYA CORPORATION, Tokyo (JP)

(21) Appl. No.: 18/580,461

(22) PCT Filed: Jul. 22, 2022
(86) PCT No.: PCT/JP2022/028468

§ 371 Date: Jul. 18, 2024

(30) Foreign Application Priority Data
Sep. 17, 2021 (JP) 2021-152269

(51) Int. Cl. A61B 1/06; A61B 1/04; G06T 3/40; G06T 7/00; G06V 10/25

(52) U.S. Cl. CPC A61B 1/0684; A61B 1/04; A61B 1/0676; G06T 3/40; G06T 7/0012; G06V 10/25; G06T 2207/10068; G06T 2207/30096

(57) ABSTRACT

A computer program causes a computer to execute processing including: acquiring an image captured by an endoscope; inputting, when the image captured by the endoscope is input, the image to a learning model learned to output a recognition result of a region of interest included in the image, and outputting a recognition result; and adjusting a light amount with which an LED provided at a distal tip of the endoscope irradiates the region of interest, on the basis of the recognition result.

### TECHNICAL FIELD

The present technology relates to a computer program, an information processing method, and an endoscope.

### BACKGROUND ART

Conventionally, there has been proposed a device that performs automatic dimming during lesion examination by an endoscope or the like. For example, a device described in Patent Literature 1 changes, by automatic dimming, luminance of a predetermined region in a medical captured image in which an observation target is captured.

### CITATION LIST

### Patent Literature

Patent Literature 1: JP 2019-42412 A

### SUMMARY OF INVENTION

### Technical Problem

However, the device described in Patent Literature 1 has a problem in that, when a region of interest suspected of being a lesion is specified by a learning model, it is not possible to improve determination accuracy by adjusting the light amount with which the region of interest is irradiated.

The present disclosure has been made in view of such circumstances, and aims at providing a computer program or the like that automatically adjusts the light amount emitted to a region of interest suspected of being a lesion specified by a learning model.

### Solution to Problem

A computer program according to one embodiment of the present disclosure causes a computer to execute processing including: acquiring an image captured by an endoscope; inputting, when the image captured by the endoscope is input, the image to a learning model learned to output a recognition result of a region of interest included in the image, and outputting a recognition result; and adjusting a light amount with which an LED provided at a distal tip of the endoscope irradiates the region of interest, on the basis of the recognition result.

In the computer program according to one embodiment of the present disclosure, the learning model outputs a likelihood that the region of interest included in the image is a lesion, and the processing includes adjusting the light amount when the likelihood is smaller than a predetermined value.

In the computer program according to one embodiment of the present disclosure, the processing includes outputting an image including the region of interest, when the likelihood that the region of interest is a lesion is smaller than a predetermined value, on a screen separate from a screen on which the image is displayed.

In the computer program according to one embodiment of the present disclosure, the processing includes switching, on a screen on which the acquired image is displayed, the image to an enlarged image obtained by enlarging the region of interest, or outputting the image including the region of interest on a screen separate from a screen on which the image is displayed, when the likelihood that the region of interest is a lesion is equal to or larger than a predetermined value.

In the computer program according to one embodiment of the present disclosure, the endoscope includes a plurality of LEDs, and the processing includes adjusting a light amount emitted by an LED corresponding to a position of the region of interest.

In the computer program according to one embodiment of the present disclosure, the processing includes receiving a change of a lower limit value or an upper limit value of the light amount with which the LED irradiates the region of interest.

In the computer program according to one embodiment of the present disclosure, the processing includes: acquiring a whiteness of the image; decreasing the light amount when the whiteness is equal to or larger than a reference value; and increasing the light amount when the whiteness is smaller than the reference value.

In the computer program according to one embodiment of the present disclosure, the processing includes changing, when the region of interest is not included in an image acquired after the light amount is adjusted, the light amount to a light amount before adjustment.

An information processing method according to one embodiment of the present disclosure includes: acquiring an image captured by an endoscope; inputting, when the image captured by the endoscope is input, the image to a learning model learned to output a recognition result of a region of interest included in the image, outputting a recognition result, and acquiring the recognition result of the region of interest included in the image from the learning model; and adjusting a light amount with which an LED provided at a distal tip of the endoscope irradiates the region of interest, on the basis of the recognition result.

An endoscope according to one embodiment of the present disclosure includes: an LED provided at a distal tip; an acquisition unit that acquires an image captured by the endoscope; an output unit that inputs, when the image captured by the endoscope is input, the image to a learning model learned to output a recognition result of a region of interest included in the image, and outputs a recognition result; and a light amount adjusting unit that adjusts a light amount with which the LED irradiates the region of interest, on the basis of the recognition result.

### Advantageous Effects of Invention

In one embodiment of the present disclosure, the light amount with which the region of interest having a low lesion likelihood is irradiated is automatically adjusted, and thus it is possible to improve the accuracy of determining a lesion.

### BRIEF DESCRIPTION OF DRAWINGS

FIG. 1 is an external view of an endoscope according to a first embodiment.

FIG. 2 is a plan view of a distal tip portion of an insertion tube.

FIG. 3 is a block diagram of an endoscope device.

FIG. 4 is an explanatory diagram related to a learning model (region-of-interest learning model).

FIG. 5 is an explanatory diagram illustrating an example of an image (control unit acquisition image) acquired from a learning model by a control unit.

FIG. 6 is an explanatory diagram illustrating an example of an LED correspondence table that represents the correspondence between a screen area and a white LED.

FIG. 7 is an explanatory diagram illustrating an example of an image (monitor image) displayed on a monitor.

FIG. 8 is an explanatory diagram illustrating an example of an image (monitor image after light amount adjustment) displayed on a monitor after the light amount is increased.

FIG. 9 is a flowchart that illustrates one example of a processing procedure executed by a control unit of a processor device.

### DESCRIPTION OF EMBODIMENTS

### First Embodiment 1

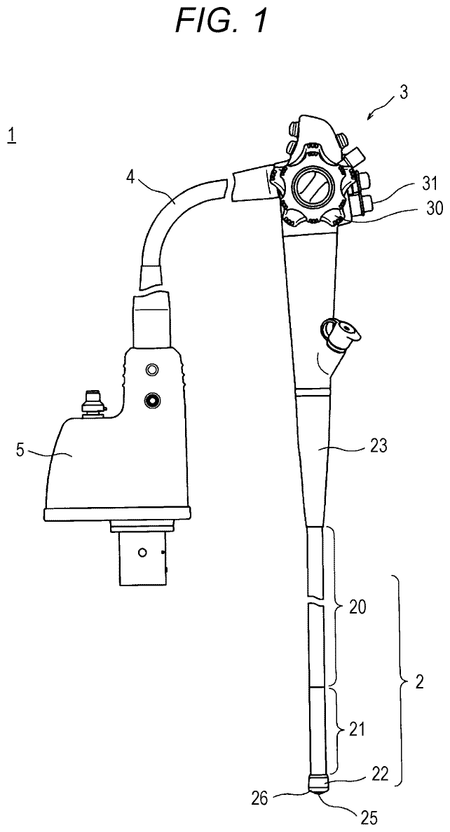

Hereinafter, the present invention will be described in detail with reference to the drawings illustrating embodiments of the present invention. FIG. 1 is an external view of an endoscope 1 according to a first embodiment. The endoscope 1 includes an insertion tube 2, an operation unit 3, a universal tube 4, and a connector unit 5. The insertion tube 2 is a portion to be inserted in a body cavity and includes a long soft portion 20, and a distal tip portion 22 connected to one end of the soft portion 20 via a bending section 21. The other end of the soft portion 20 is connected to the operation unit 3 via a cylindrical connection portion 23. One end of the universal tube 4 is connected to the operation unit 3 and extends in a direction different from the insertion tube 2, and the connector unit 5 is connected to the other end of the universal tube 4.

The operation unit 3 is provided to be gripped by a user (operator) of the endoscope 1 such as a doctor to perform various kinds of operation and includes a bending operation knob 30, a plurality of operation buttons 31, and the like. The bending operation knob 30 is connected to the bending section 21 by a wire (not illustrated) passing through the connection portion 23 and the soft portion 20. The bending section 21 is bent in two directions orthogonal to each other in an axial cross section through operation of the bending operation knob 30, thereby changing a direction of the distal tip portion 22 inserted into a body cavity.

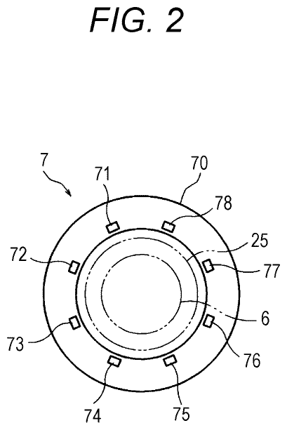

FIG. 2 is a plan view of a distal tip portion of an insertion tube. The distal tip portion 22 includes an imaging unit 6, an objective lens 25, and an illumination unit 7. The imaging unit 6 is provided inside the distal tip portion 22 to face the inner side of the objective lens 25. That is, the imaging unit 6 images a subject to be observed (an object), which is a body part such as a body cavity, through the objective lens 25. The objective lens 25 is fitted into an inner frame of a hole provided in the distal tip portion 22 of the insertion tube 2 and functions as an observation window. In FIG. 2, positions of the imaging unit 6 and the objective lens 25 are indicated by two-dot chain lines. Moreover, the illumination unit 7 is provided to face the inner side of a light distribution lens (not illustrated).

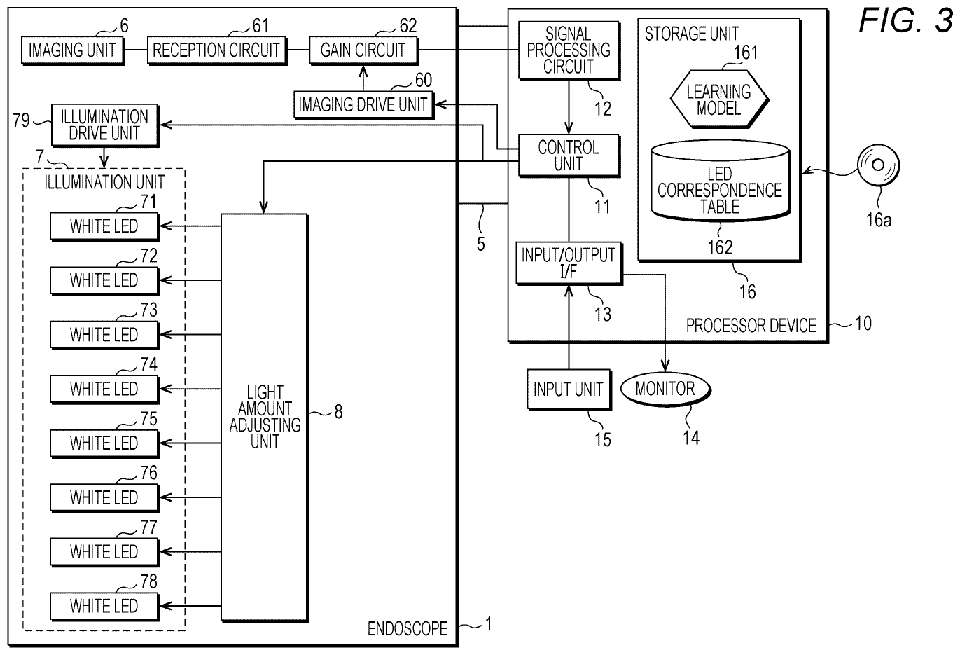

The imaging unit 6 includes an image sensor such as a complementary metal oxide semiconductor (CMOS) and an optical system for forming an image on an imaging surface of the image sensor, and images the inside of the body cavity through the objective lens 25. The objective lens 25 is, for example, a wide-angle lens, and the imaging unit 6 is configured to capture an image at a viewing angle of 180° or more by setting the optical system including the objective lens 25. The imaging unit 6 outputs, to a reception circuit 61, imaging data (image signal) of the subject to be observed (object) having been captured (see FIG. 3). The imaging data (image signal) output from the imaging unit 6 is subjected to preprocessing such as AD conversion or white balance correction in the reception circuit 61 and a gain circuit 62 (see FIG. 3), and the preprocessed imaging data is output to a signal processing circuit 12 (see FIG. 3) of a processor device 10.

The illumination unit 7 includes a substrate 70 having an annular shape surrounding the periphery of the imaging unit 6, and a plurality of white LEDs 71 to 78 mounted on one surface of the substrate 70 facing a light distribution lens 26. The white LEDs 71 to 78 are disposed at substantially equal intervals on one surface of the substrate 70 having an annular shape. The white LEDs 71 to 78 are configured by, for example, covering light emitting surfaces of blue LED chips that emit blue light with a yellow phosphor. Note that the white LED 71 may be another light emitting element such as an LD or an optical fiber. In addition, the number of white LEDs included in the endoscope is not limited to eight.

FIG. 3 is a block diagram of an endoscope device. The endoscope 1 is connected to the processor device 10 via the connector unit 5. The processor device 10 includes a control unit 11, the signal processing circuit 12, an input/output I/F 13, a storage unit 16, and the like. These control unit 11, signal processing circuit 12, input/output I/F 13, and storage unit 16 are communicably connected by an internal bus. The control unit 11 includes one or a plurality of arithmetic processing devices having a time counting function, such as central processing units (CPUs), micro-processing units (MPUs), and graphics processing units (GPUs), and performs various types of information processing, control processing, and the like related to the processor device 10 by reading and executing a program stored in the storage unit 16.

The endoscope 1 includes an imaging drive unit 60 that drives the imaging unit 6 and an illumination drive unit 79 that drives the illumination unit 7. The imaging drive unit 60 drives the imaging unit 6 in a rolling shutter type in accordance with a control command given from the control unit 11 of the processor device 10. The imaging drive unit 60 outputs an image signal to the gain circuit 62 in units of one frame via the reception circuit 61. The gain circuit 62 performs predetermined preprocessing such as white balance processing on the image signal, and outputs the preprocessed image signal as imaging data to the signal processing circuit 12 of the processor device 10.

The illumination drive unit 79 drives the illumination unit 7 in accordance with a control command given from the control unit 11 to cause the white LEDs 71 to 78 to emit light. An imaging operation of the imaging unit 6 is executed in synchronization with the driving of the illumination unit 7, and an image output obtained under illumination by the white LEDs 71 to 78 is continuously input to the signal processing circuit 12. The illumination unit 7 is switched between the driving state and the non-driving state by operating the operation button 31 provided in the operation unit 3.

The signal processing circuit 12 performs image processing, such as gamma correction and interpolation processing, on the input imaging data (image signal), and outputs the imaging data after imaging processing to the control unit 11.

The input/output I/F 13 is a communication interface that conforms, for example, to a communication standard such as USB or DSUB, and is for serially communicating with an external apparatus connected to the input/output I/F 13. A monitor 14 and an input unit 15 such as a mouse and a keyboard are connected to the input/output I/F 13. The control unit 11 performs information processing on the basis of the execution command or the event input from the input unit 15. Furthermore, the control unit 11 further performs image processing on an image output by a learning model 161 to be described later, and outputs the processed image to the external monitor 14. The monitor 14 is a display device such as a liquid crystal display or an organic EL display, and displays an image captured by the imaging unit 6 on the basis of an image signal output from the processor device 10. The user of the endoscope 1 can observe a desired site in the body cavity through the display of the monitor 14.

The endoscope 1 further includes a light amount adjusting unit 8. Similarly to the white LEDs 71 to 78, the light amount adjusting unit 8 may be mounted on the substrate 70.

The light amount adjusting unit 8 is communicably connected to the control unit 11 of the processor device 10 via the internal bus. The light amount adjusting unit 8 determines a white LED the light amount of which is to be adjusted among the white LEDs 71 to 78 on the basis of the information transmitted from the control unit 11 of the processor device 10, and increases or decreases the light amount emitted from such a white LED. The light amount to be emitted from the white LEDs 71 to 78 is adjusted by, for example, pulse width modulation (PWM) control, and the duty ratio is decreased to decrease the light amount, and the duty ratio is increased to increase the light amount. The lower limit value and the upper limit value of the light amount emitted from the white LED 71 to 78 are determined by setting the allowable width of the duty ratio. The allowable width of the duty ratio is, for example, 0.2 to 0.8, but can be changed by receiving an input at the input unit 17. In addition, the light amount of the white LED when the light amount is not adjusted is the normal light amount, and the duty ratio at this time is, for example, 0.5.

The storage unit 16 includes a volatile storage area such as a static random-access memory (SRAM), a dynamic random-access memory (DRAM), or a flash memory, and a nonvolatile storage area such as an EEPROM or a hard disk. The storage unit 16 stores in advance a program and data to be referred to at the time of processing. The program stored in the storage unit 16 may be a program that is read from a recording medium 16a readable by the control unit 11. In addition, the program may be a program that is downloaded from an external computer (not illustrated) connected to a communication network (not illustrated) and is stored in the storage unit 16.

The storage unit 16 stores an entity file (an instance file of a neural network (NN)) constituting a learning model 161 (a region-of-interest learning model) or the like to be described later. These entity files may be configured as a part of the program. Note that an information processing device connected to the processor device 10 may be separately provided, and the learning model 161 may be stored in the information processing device, so that the calculation by the learning model 161 may be performed by the information processing device. In addition, the learning model 161 may be stored in a server provided in a hospital using the endoscope 1 or a cloud server provided outside the hospital, so that the calculation by the learning model 161 may be performed by the server or the cloud server.

The storage unit 16 further stores various predetermined set values (preset data) for generating and outputting (displaying) an image. The preset data may include, for example, a normal light amount, an upper limit value or a lower limit value of the light amount emitted by the white LED 71 to 78, a constant value and a predetermined value for classifying the lesion likelihood indicating the likelihood (probability) that the region of interest is a lesion, or a reference value of the whiteness of the screen. Details of the preset data will be described later. The preset data can be changed by receiving an input at the input unit 17.

In addition, the storage unit 16 stores an LED correspondence table 162.

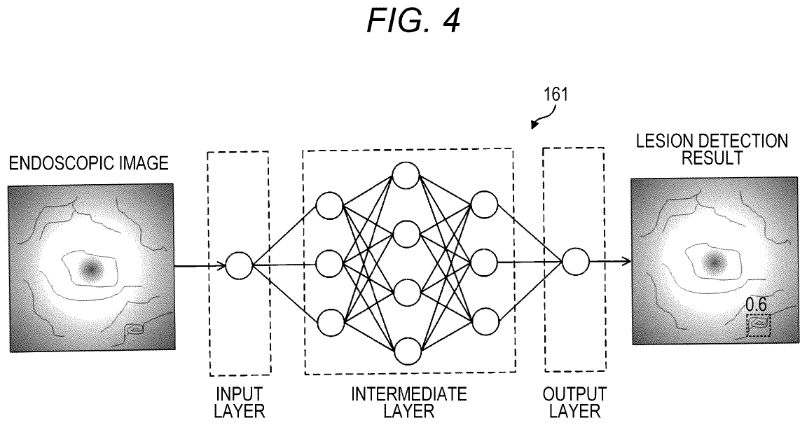

FIG. 4 is an explanatory diagram related to the learning model 161 (a region-of-interest learning model). The learning model 161 is, for example, a model that performs object detection such as regions with convolutional neural network (RCNN), Fast RCNN, Faster RCNN, single shot multibook detector (SSD), You Only Look Once (YOLO), or a neural network (NN) with a function of a segmentation network. When the learning model 161 is constituted by a neural network including a convolutional neural network (CNN) that extracts a feature amount of an image, such as an RCNN, the input layer included in the learning model 161 includes a plurality of neurons that receive an input of a pixel value of an endoscopic image, and passes the input pixel value to the intermediate layer. The intermediate layer includes a plurality of neurons that extract an image feature amount of the endoscopic image, and passes the extracted image feature amount to the output layer. The output layer includes one or a plurality of neurons that output information related to the region of interest including the position of the region of interest and the like, and outputs the position and the lesion likelihood of the region of interest on the basis of the image feature amount output from the intermediate layer. Alternatively, the learning model 161 may input the image feature amount output from the intermediate layer to a support vector machine (SVM) to perform lesion recognition. Note that the position of the region of interest is defined by image coordinate values. The neural network (learning model 161) learned using the training data is assumed to be used as a program module that is a part of artificial intelligence software. The learning model 161 is used in the control unit 11 (CPU or the like) as described above and is executed by the control unit 11 having arithmetic processing capability in this way, whereby a neural network system is configured. That is, the control unit 11 performs an arithmetic operation of extracting the feature amount of the endoscopic image input to the input layer in accordance with a command from the learning model 161 stored in the storage unit 16, and outputs the position and the lesion likelihood of the region of interest included in an image from the output layer.

As illustrated in the image of the lesion detection result in FIG. 4, the control unit 11 surrounds the region of interest with a dashed bounding box on the basis of the output of the learning model 161, and displays the lesion likelihood of the region of interest above the bounding box. In FIG. 4, the lesion likelihood is 0.6.

The learning model 161 includes the position and the lesion likelihood of the region of interest in an image as information regarding the region of interest, and outputs the information to the control unit 11. Note that, when the lesion likelihood of the region of interest is smaller than a certain value, for example, 0.2, the learning model 161 does not output the information regarding the region of interest to the control unit 11. The certain value can be changed by receiving an input at the input unit 17.

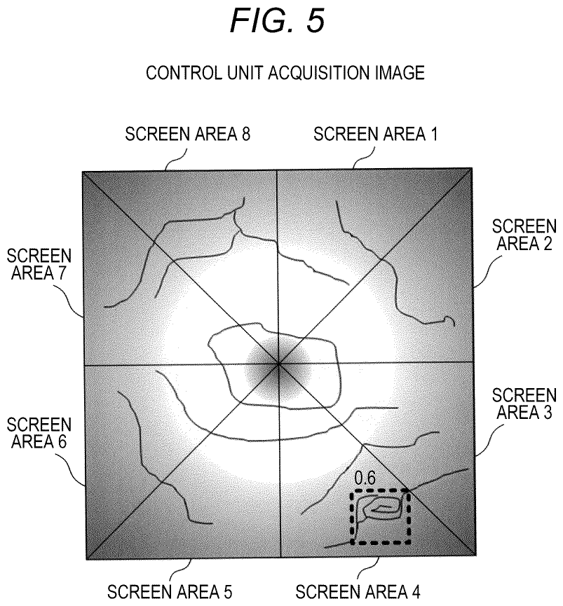

FIG. 5 is an explanatory diagram illustrating an example of an image (control unit acquisition image) acquired from a learning model by a control unit. In the image acquired from the learning model 161 by the control unit 11, the region of interest is surrounded with a dashed bounding box, and the lesion likelihood of the region of interest is displayed above the bounding box. When the lesion likelihood is smaller than a predetermined value, the control unit 11 adjusts the light amount of the white LED corresponding to the position of the region of interest. The predetermined value is, for example, 0.7, but can be changed by receiving an input at the input unit 17. A method for specifying the white LED the light amount of which is to be adjusted will be described later.

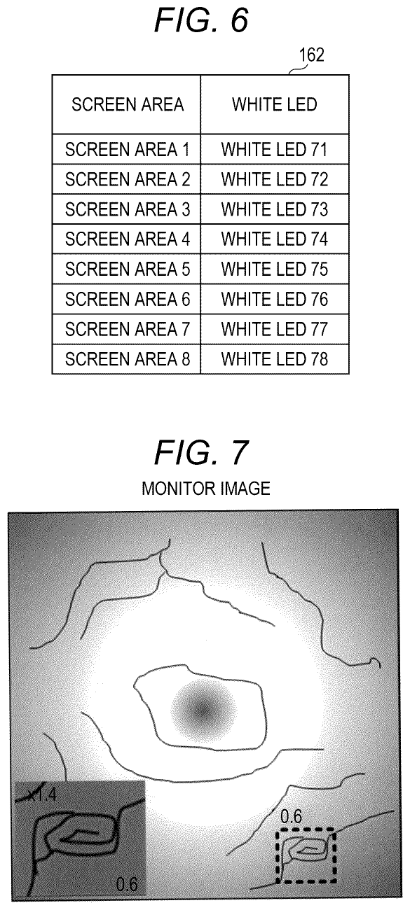

FIG. 6 is an explanatory diagram illustrating an example of an LED correspondence table that represents correspondence between a screen area and a white LED. As illustrated in FIG. 5, the image acquired by the control unit 11 is divided into eight sections (1 to 8) by 45 degrees radially from the center of the screen. As illustrated in FIG. 6, in the LED correspondence table 162, the screen areas 1 to 8 and the white LEDs 71 to 78 respectively correspond to each other, and the control unit 11 adjusts the light amount to be emitted by the white LED corresponding to the screen area including the region of interest output by the learning model 631. The area information for specifying the screen area is stored in, for example, a memory such as a RAM included in the storage unit 16, and is defined by the range of the image coordinate system in the image output by the learning model 161.

For example, when the region of interest is included in the screen area 4, as illustrated in FIG. 5, the control unit 11 adjusts the light amount emitted by the white LED 74. Furthermore, for example, when the endoscope 1 is rotated by 180 degrees about the extending direction of the insertion tube 2 in the body cavity after the control unit 11 acquires the image illustrated in FIG. 5, so that the region of interest is included in the screen area 8, the control unit 11 finishes adjustment of the light amount emitted by the white LED 74, and adjusts the light amount emitted by the white LED 78. When one region of interest is included across a plurality of screen areas, the light amounts emitted by all the LEDs corresponding to the plurality of screen areas may be adjusted, or the light amount emitted by the white LED corresponding to the screen area including the most part of the region of interest may be adjusted.

FIG. 7 is an explanatory diagram illustrating an example of an image (monitor image) displayed on a monitor. When the information regarding the region of interest (the position and the lesion likelihood of the region of interest) is included in the image acquired from the learning model 161, the control unit 11 separately displays an image including the region of interest together with the lesion likelihood on a sub-screen on the monitor 14, as illustrated in FIG. 7. Here, an image obtained by enlarging the region of interest may be displayed on the sub-screen. In this case, as illustrated in FIG. 7, the enlargement magnification may be displayed on the sub-screen. In the sub-screen of the monitor image illustrated in FIG. 7, the enlargement magnification is 1.4 times. In addition, a monitor separate from the monitor 14 may be provided to display an image including the region of interest on the monitor.

When a plurality of regions of interest are included in the image that is acquired from the learning model 161 by the control unit 11, images including respective regions of interest are displayed on a plurality of sub-screens, for example, and the color of the broken line surrounding the region of interest and the color of the frame line of the sub-screen are made same to correspond to each other.

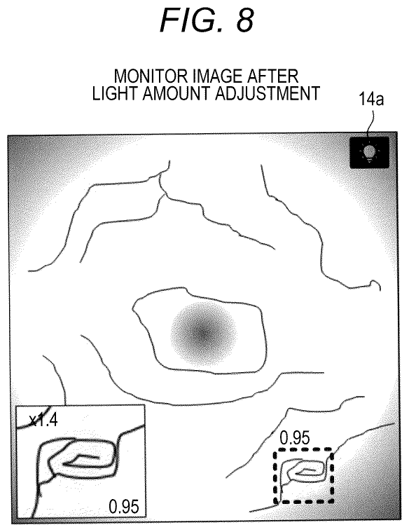

FIG. 8 is an explanatory diagram illustrating an example of an image (monitor image after light amount adjustment) displayed on a monitor after the light amount is increased. When the lesion likelihood of the region of interest included in the image acquired from the learning model 161 is smaller than a predetermined value, the control unit 11 acquires the whiteness of the image, and determines whether to decrease or increase the light amount of the white LED corresponding to the position of the region of interest on the basis of the whiteness. The whiteness is, for example, a value representing the proportion of the white portion of the endoscope screen in percentage, and is increased due to halation or the like occurred when the light amount emitted by the white LED is large.

For example, when the entire image is dark, as illustrated in FIG. 7, and the whiteness of the image is lower than a reference value, the control unit 11 increases the light amount of the white LED corresponding to the position of the region of interest, and displays the monitor image after light amount adjustment with higher whiteness as illustrated in FIG. 8 on the monitor 14. The reference value is, for example, 50, but can be changed by receiving an input at the input unit 17. For the monitor image after light amount adjustment, a light-amount-adjusted mark 14a may be displayed, as illustrated in FIG. 8.

Also when the lesion likelihood of the region of interest is higher than a predetermined value, the control unit 11 separately displays the region of interest together with the lesion likelihood on a sub-screen on the monitor 14. In the monitor image after light amount adjustment illustrated in FIG. 8, the lesion likelihood of the region of interest is 0.95.

When the whiteness of the image acquired from the learning model 161 by the control unit 11 is equal to or larger than a reference value, the control unit 11 decreases the light amount of the white LED corresponding to the position of the region of interest. Also in the case of decreasing the light amount of the white LED, the method of displaying an image on the monitor 14 is similar to the case of increasing the light amount of the white LED.

When the endoscope 1 moves in the body cavity and the region of interest is not included anymore in the image captured by the endoscope 1, the control unit 11 gradually changes the light amount of the white LED the light amount of which has been adjusted to the normal light amount.

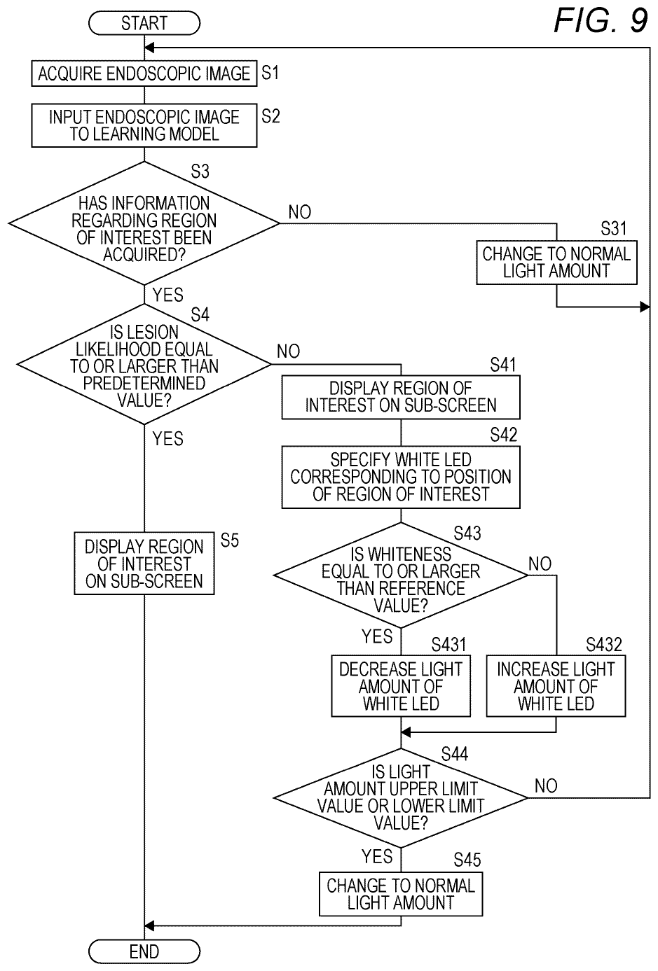

FIG. 9 is a flowchart that illustrates one example of a processing procedure executed by a control unit of a processor device. The control unit 11 of the processor device 10 starts processing of the flowchart on the basis of, for example, contents input from the input unit 17 connected to the own processor device. The light amount emitted by the white LED 71 to 78 at the start of the processing is the normal light amount.

The control unit 11 of the processor device 10 acquires an endoscopic image output from the endoscope 1 (S1). The control unit 11 may acquire the endoscopic image from the endoscope 1 in synchronization with the start of imaging of the body cavity by the endoscope 1. Furthermore, the endoscopic image may be a still image or a moving image.

The control unit 11 of the processor device 10 inputs the endoscopic image to the learning model 161 (S2). When the region of interest is included in the input endoscopic image and the lesion likelihood of the region of interest is equal to or larger than a certain value, the learning model 161 to which the endoscopic image has been input outputs the position and the lesion likelihood of the region of interest as information regarding the region of interest. When the region of interest is not included in the input endoscopic image or when the lesion likelihood of the region of interest is smaller than a certain value, the learning model 161 does not output information regarding the region of interest to the control unit 11.

The control unit 11 of the processor device 10 determines whether or not the information regarding the region of interest has been acquired from the learning model 161 (S3). When the information regarding the region of interest has not been acquired from the learning model 161 (S3: NO), the control unit 11 changes the light amount of the white LED to the normal light amount (S31). Note that, when the light amount has been the normal light amount since before the processing of S31 is executed, the normal light amount is maintained. The control unit 11 performs loop processing to execute the processing of S1 again.

When the information regarding the region of interest has been acquired from the learning model 161 (S3: YES), the control unit 11 of the processor device 10 determines whether the lesion likelihood included in the information regarding the region of interest is equal to or larger than a predetermined value (S4). When the lesion likelihood is equal to or larger than the predetermined value (S4: YES), the control unit 11 of the processor device 10 displays the region of interest on a sub-screen (S5), and finishes the processing.

When the lesion likelihood is smaller than the predetermined value (S4: NO), the control unit 11 of the processor device 10 displays the region of interest on a sub-screen (S41). The control unit 11 specifies the white LED corresponding to the position of the region of interest on the basis of the position of the region of interest included in the information regarding the region of interest and the LED correspondence table (S42). The white LED specified at this time is a white LED the light amount of which is to be adjusted.

The control unit 11 of the processor device 10 determines whether or not the acquired whiteness of the endoscope screen is equal to or larger than a reference value (S43). The control unit 11 of the processor device 10 decreases the light amount emitted by the white LED specified at S42 (S431) when the whiteness of the endoscope screen is equal to or larger than the reference value (S43: YES), and increases the light amount emitted by the white LED specified at S42 (S432) when the whiteness is smaller than the reference value (S43: NO). For example, the control unit 11 gradually decreases the duty ratio output to the white LED from 0.5 to 0.2 in the case of decreasing the light amount emitted by the white LED, and gradually increases the duty ratio output to the white LED from 0.5 to 0.8 in the case of increasing the light amount.

The control unit 11 of the processor device 10 determines whether or not the light amount emitted by the white LED is the upper limit value or the lower limit value on the basis of the duty ratio output to the white LED (S44). When the light amount is the upper limit value or the lower limit value (S44: YES), the control unit 11 changes the light amount emitted by the white LED to the normal light amount (S45), and finishes the processing. When the light amount is not the upper limit value or the lower limit value (S44: NO), the control unit 11 performs loop processing to execute the processing of S1 again.

In the present embodiment, the light amount with which the region of interest having a low lesion likelihood is irradiated is automatically adjusted, and thus it is possible to improve the accuracy of determining a lesion.

### Modification

In the above-described embodiment, the position of the region of interest is defined by the image coordinate values, but the position of the region of interest may be defined by the pixel number. In this case, the screen area is defined by a range of pixel numbers.

Furthermore, in the case of the data in the image arrangement format output by the learning model 161, the position of the region of interest may be defined by the sequence number, and in this case, the screen area is defined by a range of sequence numbers.

In the above-described embodiment, when the lesion likelihood of the region of interest included in the acquired endoscopic image is equal to or larger than the predetermined value, it is displayed on a sub-screen. However, as an alternative of display on the sub-screen, the region of interest having a lesion likelihood equal to or larger than the predetermined value may be enlarged and displayed. In addition, the region of interest may be enlarged and displayed on a sub-screen.

In the above-described embodiment, whether to decrease or increase the light amount emitted by the white LED is determined by determining whether the whiteness of the screen is equal to or larger than the reference value. However, a photometric circuit may be provided in the endoscope, and whether to decrease or increase the light amount emitted by the white LED may be determined on the basis of the reflected light amount in the body cavity that is measured by the photometric circuit.

The embodiments disclosed herein are exemplary in all respects, and it should be considered that the embodiments are not restrictive. The technical features described in the embodiments can be combined with each other, and the scope of the present invention is intended to include all modifications within the scope of the claims and the scope equivalent to the claims.

### REFERENCE SIGNS LIST

1 endoscope
2 insertion tube
20 soft portion
21 bending section
22 distal tip portion
23 connection portion
25 objective lens
26 light distribution lens
3 operation unit
31 operation button
4 universal tube
5 connector unit
6 imaging unit
60 imaging drive unit
61 reception circuit
62 gain circuit
7 illumination unit
70 substrate
71 to 78 white LED
79 illumination drive unit
8 light amount adjusting unit
10 processor device
11 control unit
12 signal processing circuit
13 input/output I/F
14 monitor
15 input unit
16 storage unit
161 learning model
16a recording medium

1. A non-transitory computer-readable storage medium that stores an executable computer program for causing a computer to execute processing comprising:
acquiring an image captured by an endoscope;
inputting, when the image captured by the endoscope is input, the image to a learning model learned to output a recognition result of a region of interest included in the image, and outputting a recognition result; and
adjusting a light amount with which an LED provided at a distal tip of the endoscope irradiates the region of interest, on the basis of the recognition result.

2. The storage medium according to claim 1, wherein
the learning model outputs a likelihood that the region of interest included in the image is a lesion, and
the processing includes adjusting the light amount when the likelihood is smaller than a predetermined value.

3. The storage medium according to claim 1, wherein
the processing includes outputting an image including the region of interest, when the likelihood that the region of interest is a lesion is smaller than a predetermined value, on a screen separate from a screen on which the image is displayed.

4. The storage medium according to claim 1, wherein
the processing includes switching, on a screen on which the acquired image is displayed, the image to an enlarged image obtained by enlarging the region of interest, or outputting the image including the region of interest on a screen separate from a screen on which the image is displayed, when the likelihood that the region of interest is a lesion is equal to or larger than a predetermined value.

5. The storage medium according to claim 1, wherein
the endoscope includes a plurality of LEDs, and
the processing includes adjusting a light amount emitted by an LED corresponding to a position of the region of interest.

6. The storage medium according to claim 1, wherein
the processing includes receiving a change of a lower limit value or an upper limit value of the light amount with which the LED irradiates the region of interest.

7. The storage medium according to claim 1, wherein the processing includes:
acquiring a whiteness of the image;
decreasing the light amount when the whiteness is equal to or larger than a reference value; and
increasing the light amount when the whiteness is smaller than the reference value.

8. The storage medium according to claim 1, wherein
the processing includes changing, when the region of interest is not included in an image acquired after the light amount is adjusted, the light amount to a light amount before adjustment.

9. An information processing method, comprising:
acquiring an image captured by an endoscope;
inputting, when the image captured by the endoscope is input, the image to a learning model learned to output a recognition result of a region of interest included in the image, outputting a recognition result, and acquiring the recognition result of the region of interest included in the image from the learning model; and
adjusting a light amount with which an LED provided at a distal tip of the endoscope irradiates the region of interest, on the basis of the recognition result.

10. An endoscope, comprising:
an LED provided at a distal tip;
an acquisition unit that acquires an image captured by the endoscope;
an output unit that inputs, when the image captured by the endoscope is input, the image to a learning model learned to output a recognition result of a region of interest included in the image, and outputs a recognition result; and
a light amount adjusting unit that adjusts a light amount with which the LED irradiates the region of interest, on the basis of the recognition result.
